(19) United States
(12) Patent Application Publication
(10) Pub. No.: US 2011/0029051 A1
(43) Pub. Date: Feb. 3, 2011

(54) Upper body thermal relief apparatus and method

(76) Inventor: Leslie Ann Ross, Rockwall, TX (US)

Correspondence Address: Michael G. Cameron, 2025 Savannah Drive, McKinney, TX, 75070, US

(21) Appl. No.: 12/462,304

(22) Filed: Aug. 3, 2009

(51) Int. Cl. A61F 7/10

(52) U.S. Cl. 607/108

(57) ABSTRACT

An apparatus and method for relieving nausea being an enclosure formed from a plurality of layers of single sheets being joined along the periphery thereof. The enclosure has a main quadrilateral portion with a left elongated extension extending from the top of the left side of the main quadrilateral portion and a right elongated extension extending from the top of the right side of the main quadrilateral portion. The left elongated extension and right elongated extension each have a mating mechanism adapted to be joined at the ends thereof and coupled to encircle a neck. The main quadrilateral portion opening has a closure flap. At least one cold pack is dimensioned to fit within the enclosure through the main quadrilateral portion opening.

### CROSS REFERENCE TO RELATED APPLICATION

Not Applicable

### FIELD OF THE INVENTION

The present invention relates to a therapeutic apparatus and method for relieving nausea and heat discomforts by the application of a cold source proximate the upper torso of a body.

### BACKGROUND

It is well known to apply cold packs directly to parts of the body to relieve muscle stress and strain. What is desired is a cold pack tailored for placement around the upper torso below the neck to relieve the effects of nausea and heat stress. With respect to muscle aches and pains, the application of cold narrows the blood vessels, limiting the blood flow to the injury and providing temporary relief.

### SUMMARY OF THE INVENTION

The present invention relates to non-invasive apparatus and method to relieve, inter alia, bodily discomforts such as nausea and heat stress. To those skilled in the art to which this invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### DESCRIPTION OF THE DRAWINGS

A more complete understanding of the present invention may be obtained by reference to the following Detailed Description, when taken in conjunction with the accompanying Drawings, wherein:

FIG. 1 is a front perspective view of first embodiment of the present invention having a vertical pocket aperture;

FIG. 2 is a front perspective view of first embodiment of the present invention having a horizontal pocket aperture;

FIG. 3 is a rear perspective view of the first embodiment of the present invention;

FIG. 4 is a cross-sectional view of the front of a first layered design of the present invention;

FIG. 5 is a top view of the first embodiment of the present invention;

FIG. 6 is a front perspective view of second embodiment of the present invention;

FIG. 7 is a rear perspective view of the second embodiment of the present invention;

FIG. 8 is a cross-sectional view of the front of a second layered design of the present invention;

FIG. 9 is a top view of the second embodiment of the present invention;

FIG. 10 is a third embodiment of the present invention; and

FIG. 11 is a fourth embodiment of the present invention.

### DETAILED DESCRIPTION

The present invention will now be described more fully with reference to the accompanying drawings, in which several embodiments of the invention is shown. Each embodiment can further include a different layering of materials used to construct the invention. This invention may, however, be embodied in many different forms and should not be constructed as limited to the embodiments set forth herein; rather, these embodiments are provided so that this disclosure will be thorough and complete, and will fully convey the scope of the invention to those skilled in the art.

Generally, one embodiment of the present invention is a specially dimensioned enclosure adapted to hold a cold pack against the front, upper torso of a user. Another embodiment of the present invention is a specially dimensioned enclosure adapted to hold a first cold pack against the front, upper torso and a second cold pack against the upper back of a user.

An aspect of the present invention uses a cold pack in an enclosure having a thermally transparent inner layer and insulating outer layer. Another aspect of the present invention uses a cold pack in an enclosure having a thermally transparent inner layer and outer layer. A cold pack is an enclosed container holding a cold source of, inter alia, a frozen water based fluid, refrigerant gel or liquid, or frozen beads or organic material. The refrigerant is non-toxic and absorbs a considerable amount of heat, as its specific heat capacity is high. Other cold sources include non-toxic materials such as hydroxyethyl cellulose, vinyl-coated silica gel and chemical slurries which have a freezing point between zero and 32 degrees Fahrenheit. A slurry which may be employed is a mixture of 10% isopropyl alcohol and water which creates a semi-pliable slurry at a temperature of 20 degrees Fahrenheit. Another type of cold pack has a cold source that uses the endothermic reaction of ammonium nitrate and water to cool down quickly. When a user breaks a tube inside the cold pack, the ammonium nitrate is released allowing it to mix with the water. Other chemicals which produce a similar effect include calcium chloride and ammonium chloride. The cold pack used in the present invention is non-toxic and hypoallergenic. For example, in one aspect of the present invention a silicate gel filler and flexible vinyl covering are used in the cold pack.

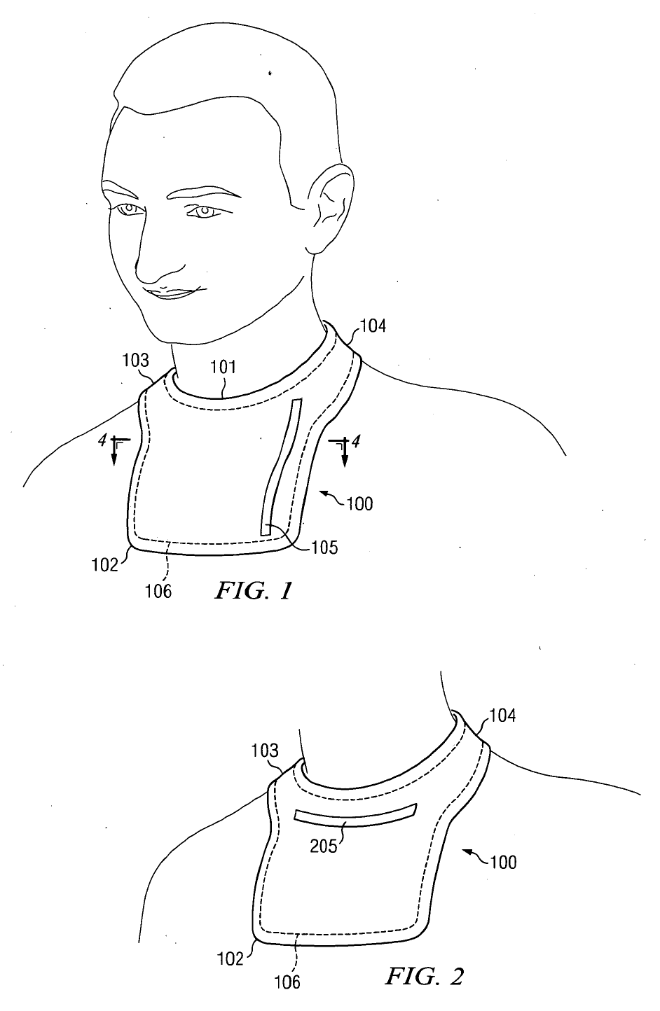

The first embodiment of the apparatus of the present invention is adapted primarily for relieving nausea. Such apparatus 100 can be seen in FIGS. 1 to 5. Referring now to FIG. 1, there is shown a front perspective view of first embodiment of the present invention having a vertical pocket aperture. As seen therein, an enclosure 100 is formed from a plurality of layers of sheets of material being joined along the periphery thereof. Enclosure 101 has a main quadrilateral portion 102 with a left elongated extension 103 extending from the top of the left side of the main quadrilateral portion 102 and a right elongated extension 104 extending from the top of the right side of the main quadrilateral portion 102. The left elongated extension 103 and right elongated extension 104 each having a mating mechanism adapted to be joined at the ends thereof and coupled to encircle a neck. A main quadrilateral portion opening having a closure flap 105 is seen in FIG. 1 as vertical pocket aperture. At least one cold pack 106 is dimensioned to fit within the enclosure through the main quadrilateral portion opening 105.

Referring now to FIG. 2, there is shown a front perspective view of first embodiment of the present invention having a closure flap 205 in the form of a horizontal pocket aperture.

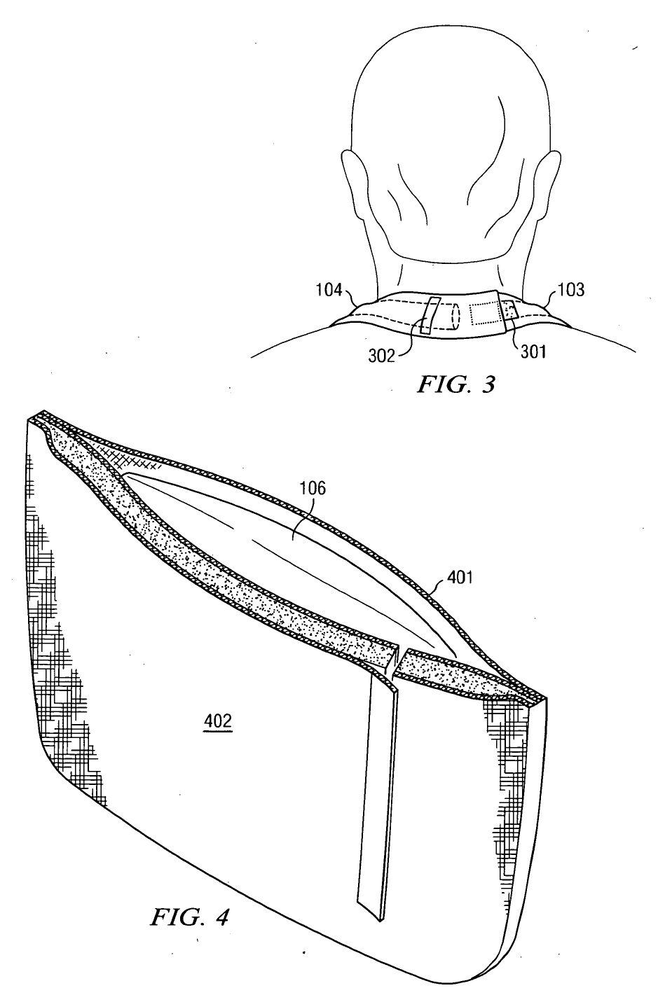

Referring now to FIG. 3, there is shown a rear perspective view of the first embodiment of the present invention showing the mating mechanism 301 such as a strip of hook and loop material, such as Velcro® and an extension opening 302 having an closure flap on the right elongated extension 104, however, such extension opening 302 having a closure flap can also be located on the left elongated extension 103. Further, any other suitable mechanism other than a hook and loop material can be used, such as a snap, zipper, or button and button hole arrangement.

The cold pack used in the enclosure can be a single cold pack dimensioned to fit in the main quadrilateral portion 102 and the right elongated extension 104 and left elongated extensions 103, or a plurality of cold packs, one to fit in the main quadrilateral portion 102 and one to fit in the right elongated extension 104, across the interior of the top of the main quadrilateral portion 102 and the left elongated extension 103. For example, in the latter aspect of the present invention, the first cold pack is formed of two layered quadrilateral sheets coupled along the periphery to form a volume and adapted to hold a cold source, the cold pack dimensioned to fit within the main quadrilateral portion of the enclosure through the main quadrilateral portion opening with a second cold pack being formed of two long rectangular sheets being coupled along the periphery thereof to form a generally cylindrical volume and adapted to hold a cold source, the second cold pack being dimensioned to fit from the end of the left extension, across the top of the main quadrilateral portion of the enclosure to the end of the right extension. The second cold pack is inserted through the extension opening.

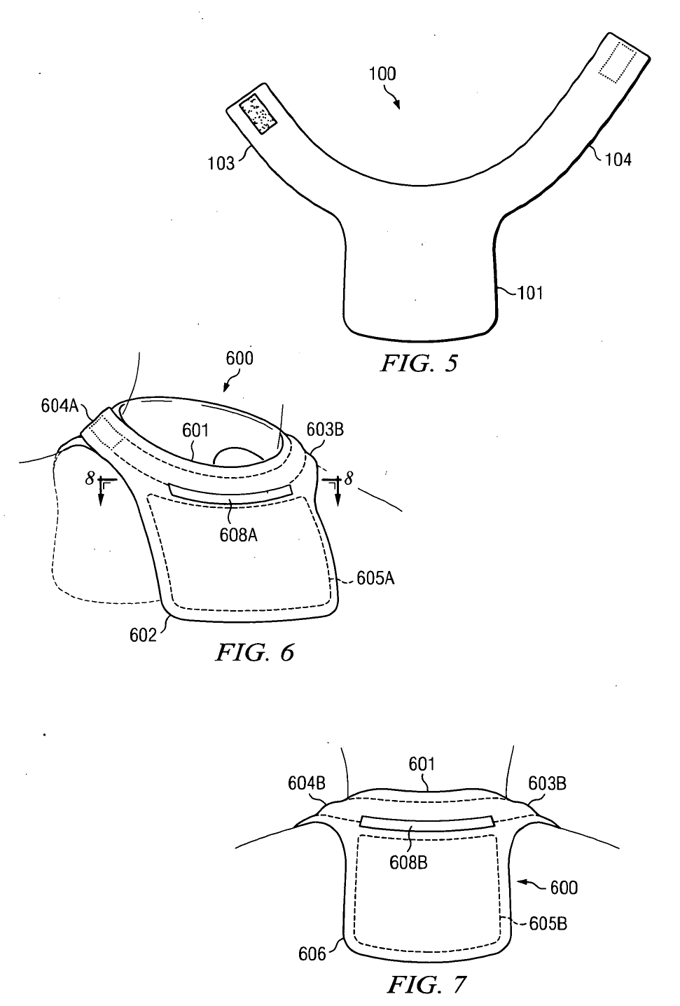

Referring now to FIG. 4, there is shown a cross-sectional view of the front of a first layered design of the present invention. As seen, there are a plurality of layers of sheets including an inner layer 401 adapted to be placed against the upper torso of a body and an outer layer 402 opposite the inner layer 401. Within the layers, in the formed pocket of the enclosure is a cold pack 106. The inner layer 401 is a somewhat thermally transparent material for transmitting heat from the upper front torso of a body to the cold pack 106, the material further adapted to wick away and retain moisture from the cold pack 106 and the upper body of the user. Such material of the inner layer 401 comprises at least one selected from the group consisting of cloth, knitted fabric, felt, chamois, terry cloth, natural textile, artificial textile, polyester, or rayon. The outer layer 402 can be an insulating material such as neoprene, foamed rubber, thin flexible styrofoam, high density open cell polyurethane foam, or collapsible foam. A covering can be added to the insulating material, such as a denier covering. The cold pack 106 can be a gel pack having a non-toxic gel serving as the cold source and coupled, flexible, laminate sheets containing the gel. Such gel pack can further be compartmentalized longitudinally to allow greater flexibility when frozen. Referring now to FIG. 5, there is shown a top view 100 of the first embodiment of the present invention. As seen therein, adjustable Velcro® closures retain the enclosure of the present invention in place encircling the neck of the user.

FIGS. 6-9 illustrate a second embodiment of the present invention used to relieve heat stress. Referring now to FIG. 6, there is shown a front perspective view of second embodiment of the present invention. FIG. 7, illustrates a rear perspective view of the second embodiment, of the present invention. The second embodiment is an apparatus 600 being an enclosure 601 formed from a plurality of layers of sheets of material being joined along the periphery thereof. The enclosure 601 has a front quadrilateral portion 602 with a left elongated extension 604A extending from the top of the left side of the front quadrilateral portion 602 and a right elongated extension 603B extending from the top of the right side of the front quadrilateral portion 602. As seen in FIG. 7, there is further a rear quadrilateral portion 606 with a left elongated extension 603A extending from the top of the left side of the rear quadrilateral portion 606 and a right elongated extension 604B extending from the top of the right side of the rear quadrilateral portion 606. The right elongated extension 603B of the front quadrilateral portion 602 and the left elongated extension 604B of rear quadrilateral portion 606 being formed as a continuous portion. The left elongated extension 604A of the front quadrilateral portion 602 and the right elongated extension 603A of the rear quadrilateral portion 606 each having a mating mechanism 607A, 607B adapted to be joined at the ends thereof and coupled to encircle a neck. Such a mating mechanism can be a hook and loop fastener, for example, Velcro®. Further, any other suitable mechanism other than a hook and loop material can be used, such as a snap, zipper, or button and button hole arrangement.

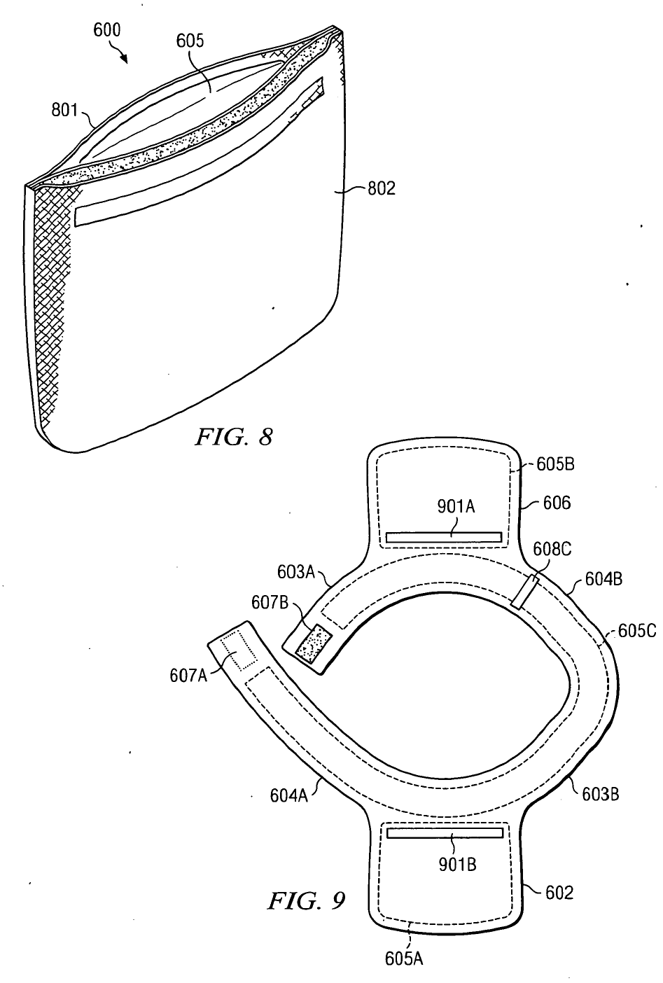

Referring now to FIG. 8, there is shown a cross-sectional view of the front of a second layered design of the present invention showing the inner layer 801 of the enclosure 600, cold pack 605 and outer layer 802 of the enclosure 600. The second embodiment includes at least one cold pack 605 dimensioned to fit within the enclosure 600 through an opening 608. There can further be a plurality of cold packs, adapted to be enclosed through a front quadrilateral portion opening 608A having a closure flap, a rear quadrilateral portion opening 608B having a closure flap. Further, in another aspect there can be an extension opening 608C having a closure flap. The first cold pack 605A is formed of two layered quadrilateral sheets coupled along the periphery forming a volume and adapted to hold a cold source. It is dimensioned to fit within the front quadrilateral portion 602 of the enclosure through the front quadrilateral portion opening 608A. A second cold pack 605B is formed of two layered quadrilateral sheets coupled along the periphery forming a volume and adapted to hold a cold source. The second cold pack 605B is dimensioned to fit within the rear quadrilateral portion 606 of the enclosure through the rear quadrilateral portion opening 608B. Such first and second cold packs can further be compartmentalized longitudinally to allow greater flexibility when frozen. A third cold pack 605C is formed of two long rectangular sheets coupled along the periphery thereof to form a generally cylindrical volume and adapted to hold a cold source. As seen in the top view of FIG. 9, the third cold pack 605C is dimensioned to fit from the end of the left elongated extension 604A of the front quadrilateral portion 602, across the top of the front quadrilateral portion 602 of the enclosure through the right elongated extension 603B of the front quadrilateral portion 602 and left elongated extension 604B of the rear quadrilateral portion 606 across the top of the rear quadrilateral portion 606 and through the right elongated extension 603A of the rear quadrilateral portion 606, the third cold pack 605C being inserted through the extension opening 608C.

The plurality of layers include an inner layer 801 adapted to be placed against the upper torso of a body and an outer layer opposite the inner layer 801. The inner layer 801 comprises a material for transferring heat from the upper torso of the body to the cold pack 605 and wick away and retain moisture from the cold pack 605 and the upper body of the user. The material of the inner layer 801 comprises at least one selected from the group consisting of cloth, knitted fabric, felt, chamois, terry cloth, natural textile, artificial textile, polyester, rayon and velvet. The mating mechanism 607A, 607B of the left elongated extension 604A of the front quadrilateral portion 602 and the right elongated extension 603B of the rear quadrilateral portion 606 comprise a strip of hook and loop closure means such as Velcro®, thus providing an adjustable coupling means. As in the first embodiment, the cold pack can include a gel pack having a non-toxic gel serving as the cold source and coupled, flexible, laminate sheets containing the gel.

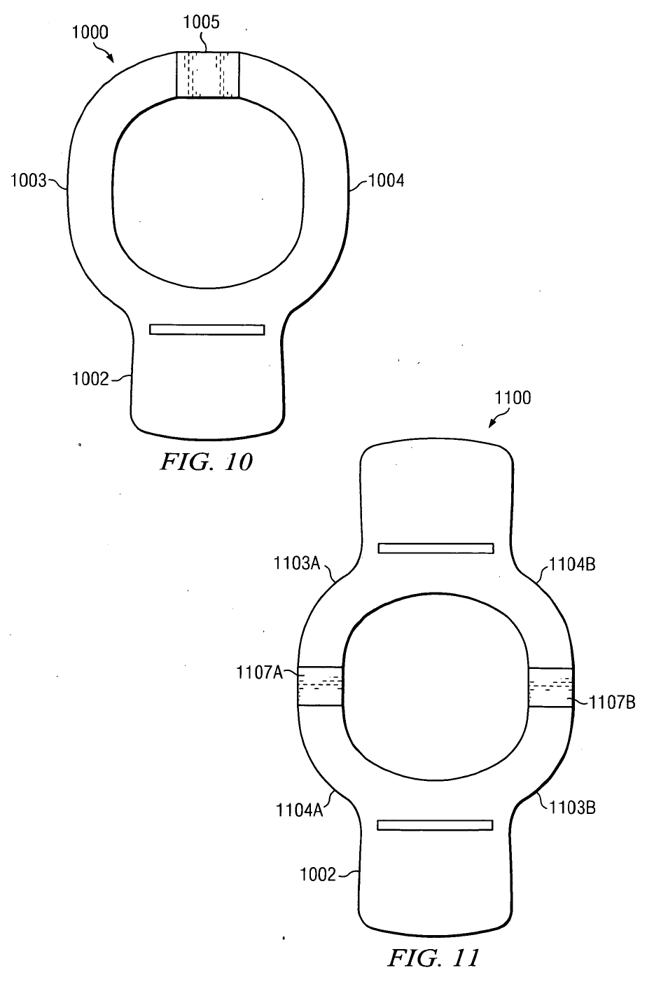

FIG. 10 is a third embodiment of the present invention. The apparatus 1000 seen in FIG. 10 is similar to that of FIGS. 1-5, however, as seen therein, the left elongated extension 1003 extending from the top of the left side of the main quadrilateral portion 1002 and a right elongated extension 1004 extending from the top of the right side of the main quadrilateral portion 1002 are joined by at least one elastic portion 1005 forming a continuous loop adapted to expand to fit over a head and then encircle a neck. As one skilled in the art would recognize, one or more elastic portions can be spaced along the elongated extensions.

FIG. 11 is a fourth embodiment of the present invention. The apparatus 1100 seen in FIG. 11 is similar to that of FIGS. 6-9, however, as seen therein, the front quadrilateral portion's left elongated extension 1104A extending from the top of the left side of the front quadrilateral portion 1102 and right elongated extension 1103B extending from the top of the right side of the front quadrilateral portion 1102, are joined by at least one elastic portion 1107A, 1107B, respectively, with the rear quadrilateral portion's left elongated extension 1103A extending from the top of the left side of the rear quadrilateral portion 1106 and right elongated extension 1104B extending from the top of the right side of the rear quadrilateral portion 1106, thus forming a continuous loop adapted to expand to fit over a head and then encircle a neck. As one skilled in the art would recognize, one or more elastic portions can be spaced along the elongated extensions.

The present invention further comprises a method of relieving nausea, comprising the steps of providing an substantially quadrilateral enclosure having a right elongated extension and left elongated extension, inserting a cold pack inside the enclosure, placing the enclosure against the upper, front torso of a body, and coupling the right elongated extension to the left elongated extension with a coupling means around a neck.

The previous description is of the preferred embodiments of the invention are not limited by this description. The scope of the present invention is instead defined by the following claims.

I claim:

1. An apparatus for relieving nausea, comprising:
an enclosure formed from a plurality of layers of single sheets being joined along the periphery thereof;
the enclosure having a main quadrilateral portion with a left elongated extension extending from the top of the left side of the main quadrilateral portion and a right elongated extension extending from the top of the right side of the main quadrilateral portion;
the left elongated extension and right elongated extension each having a mating mechanism adapted to be joined at the ends thereof and coupled to encircle a neck;
a main quadrilateral portion opening having a closure flap; and
at least one cold pack dimensioned to fit within the enclosure through the main quadrilateral portion opening.

2. The apparatus of claim 1, further comprising:
an extension opening having an closure flap;
a first cold pack being formed of two layered quadrilateral sheets coupled along the periphery to form a volume and adapted to hold a cold source, the first cold pack dimensioned to fit within the main quadrilateral portion of the enclosure through the main quadrilateral portion opening; and
a second cold pack being formed of two long rectangular sheets being coupled along the periphery thereof to form a generally cylindrical volume and adapted to hold a cold source, the second cold pack being dimensioned to fit from the end of the left extension, across the top of the main quadrilateral portion of the enclosure to the end of the right extension, the second cold pack being inserted through the extension opening.

3. The apparatus of claim 2, wherein the plurality of layers of sheets include an inner layer adapted to be placed against the upper torso of a body and an outer layer opposite the inner layer.

4. The apparatus of claim 3, wherein the inner layer comprises a somewhat thermally transparent material for transmitting heat from the upper front torso of a body to the cold pack, the material further adapted to wick away and retain moisture from the cold pack.

5. The apparatus of claim 4, wherein the material of the inner layer comprises at least one selected from the group consisting of cloth, knitted fabric, felt, chamois, terry cloth, natural textile, artificial textile, polyester and rayon.

6. The apparatus of claim 3, wherein the outer layer comprises an insulating material.

7. The apparatus of claim 6, wherein the insulating material comprises one selected from the group consisting of neoprene, foamed rubber, thin flexible styrofoam, high density open cell polyurethane foam, and collapsible foam.

8. The apparatus of claim 7, further comprising a covering on the insulating material, further comprising a denier covering.

9. The apparatus of claim 2, wherein the cold pack comprises a gel pack having a non-toxic gel serving as the cold source and coupled, flexible, laminate sheets containing the gel.

10. The apparatus of claim 2, wherein the main quadrilateral portion closure flap and extension opening closure flap further include a hook and loop closure means such as Velcro®.

11. The apparatus of claim 2, wherein the mating mechanism of the left elongated extension and right elongated extension comprise a strip of hook and loop closure means such as Velcro®, thus providing an adjustable coupling means.

12. An apparatus for relieving heat stress, comprising:
an enclosure formed from a plurality of layers of sheets being joined along the periphery thereof;
the enclosure having a front quadrilateral portion with a left elongated extension extending from the top of the left side of the front quadrilateral portion and a right elongated extension extending from the top of the right side of the front quadrilateral portion;
a rear quadrilateral portion with a left elongated extension extending from the top of the left side of the rear quadrilateral portion and a right elongated extension extending from the top of the right side of the rear quadrilateral portion;
the right elongated extension of the front quadrilateral portion and the left elongated extension of rear quadrilateral portion being formed as a continuous portion;
the left elongated extension of the front quadrilateral portion and the right elongated extension of the rear quadrilateral portion each having a mating mechanism adapted to be joined at the ends thereof and coupled to encircle a neck; and
at least one cold pack dimensioned to fit within the enclosure through an opening.

13. The apparatus of claim 12, further comprising:
a front quadrilateral portion opening having a closure flap;
a rear quadrilateral portion opening having a closure flap;
an extension opening having a closure flap;
a first cold pack being formed of two layered quadrilateral sheets coupled along the periphery forming a volume and adapted to hold a cold source, the first cold pack dimensioned to fit within the front quadrilateral portion of the enclosure through the front quadrilateral portion opening;
a second cold pack being formed of two layered quadrilateral sheets coupled along the periphery forming a volume and adapted to hold a cold source, the second cold pack dimensioned to fit within the rear quadrilateral portion of the enclosure through the rear quadrilateral portion opening; and
a third cold pack being formed of two long rectangular sheets being coupled along the periphery thereof to form a generally cylindrical volume and adapted to hold a cold source, the third cold pack being dimensioned to fit from the end of the left elongated extension of the front quadrilateral portion, across the top of the front quadrilateral portion of the enclosure through the right elongated extension of the front quadrilateral portion and left elongated extension of the rear quadrilateral portion across the top of the rear quadrilateral portion and through the right elongated extension of the rear quadrilateral portion, the third cold pack being inserted through the extension opening.

14. The apparatus of claim 13, wherein the plurality of layers include an inner layer adapted to be placed against the upper torso of a body and an outer layer opposite the inner layer.

15. The apparatus of claim 13, wherein the inner layer comprises a material for transferring heat from the upper torso of the body to the cold pack and wick away and retain moisture from the cold pack.

16. The apparatus of claim 15, wherein the material of the inner layer comprises at least one selected from the group consisting of cloth, knitted fabric, felt, chamois, terry cloth, natural textile, artificial textile, polyester and rayon.

17. The apparatus of claim 12, wherein the mating mechanism of the left elongated extension of the front quadrilateral portion and the right elongated extension of the rear quadrilateral portion comprise a strip of hook and loop closure means such as Velcro®, thus providing an adjustable coupling means.

18. The apparatus of claim 13, wherein the cold pack comprises a gel pack having a non-toxic gel serving as the cold source and coupled, flexible, laminate sheets containing the gel.

19. A method of relieving nausea, comprising the steps of:
providing an substantially quadrilateral enclosure having a right elongated extension and left elongated extension;
inserting a cold pack inside the enclosure;
placing the enclosure against the upper, front torso of a body; and
coupling the right elongated extension to the left elongated extension with a coupling means around a neck.

20. The method of claim 19, wherein the cold pack comprises a gel pack having a non-toxic gel as the cold source and coupled, flexible, laminate sheets containing the gel.
